(19) United States
(12) Patent Application Publication
(10) Pub. No.: US 2026/0020679 A1
(43) Pub. Date: Jan. 22, 2026

(54) GROUNDING FOOT REST

(71) Applicant: Ermelinda HYSENBEGASI, Nice (FR)

(72) Inventor: Ermelinda HYSENBEGASI, Nice (FR)

(73) Assignee: ERMELINDA HYSENBEGASI, Nice (FR)

(21) Appl. No.: 19/270,751

(22) Filed: Jul. 16, 2025

Related U.S. Application Data
(60) Provisional application No. 63/672,764, filed on Jul. 18, 2024.

(51) Int. Cl. A47C 16/02; A61N 1/14

(52) U.S. Cl. CPC A47C 16/02; A61N 1/14

(57) ABSTRACT

The present invention relates to a grounding foot rest that combines feet support with an integrated grounding mechanism to establish a connection between the user's bare feet and the Earth's natural electric field. The device includes a supportive platform with a conductive contact surface and a grounding pathway, allowing for passive electrical connection during seated activities. The design promotes posture, comfort, and the potential benefits of grounding in indoor environments.

### BACKGROUND OF THE INVENTION

Extended periods of sitting have been linked to poor posture, discomfort, and reduced circulation. Conventional footrests can help encourage better posture and leg positioning but do not address the emerging wellness practice of grounding (also known as earthing). Some studies suggest that grounding “the practice of connecting the human body to the Earth's surface electrons” may support general wellness. Accordingly, there remains a need for an ergonomic footrest that incorporates grounding functionality to help users maintain natural electrical contact with the Earth while seated indoors for extended periods.

### SUMMARY OF THE INVENTION

This invention provides a dual function foot rest that supports the feet promoting ergonomic posture and comfort while delivering the potential wellness benefits associated with grounding. It includes adjustable height, tilt or flat options, dynamic movement capabilities, and an integrated conductive cover that facilitates electron transfer when connected to the Earth. The foot rest may also function as a backrest or travel pillow, enhancing comfort and versatility. While one embodiment resembles a half-moon or semi-cylinder with a curved top and a rectangular base, other shapes may be used to achieve the same support for the feet and grounding functions.

### BRIEF DESCRIPTION OF FIGURES

FIG. 1 is a perspective view of the grounding foot rest in an assembled configuration.

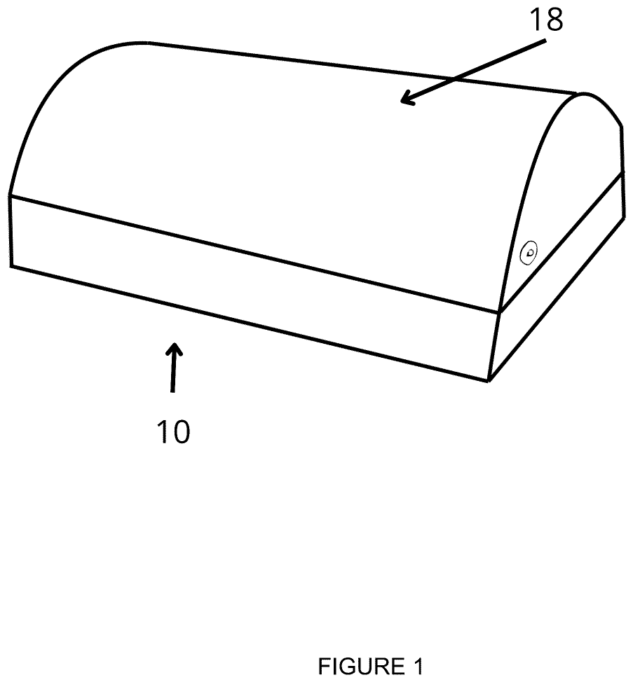

FIG. 2 is a side and front view illustrating the curved top surface, rectangular base component, and adjustable height mechanism.

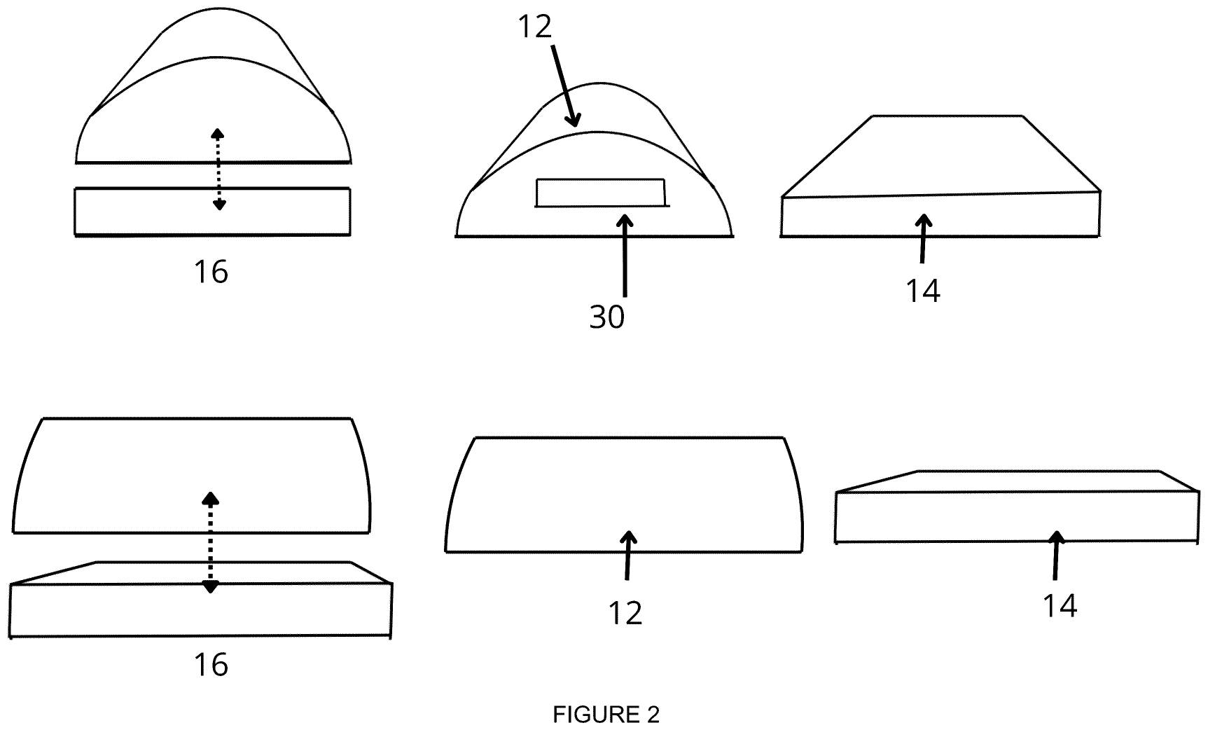

FIG. 3 is a view of the base of the grounding foot rest.

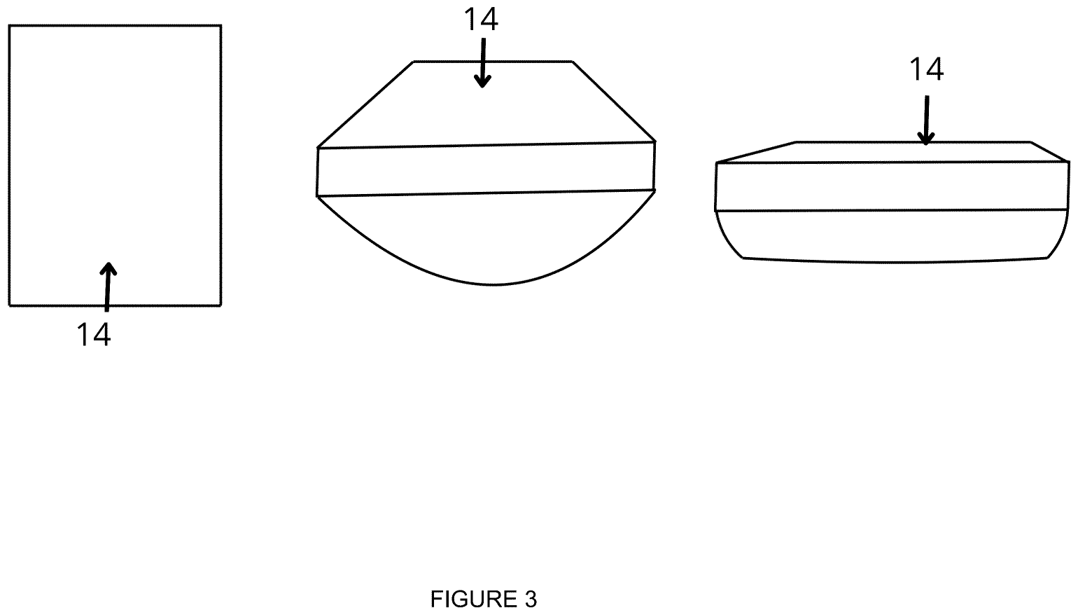

FIG. 4 is a view showing the detachable grounding wire and grounding plug connection.

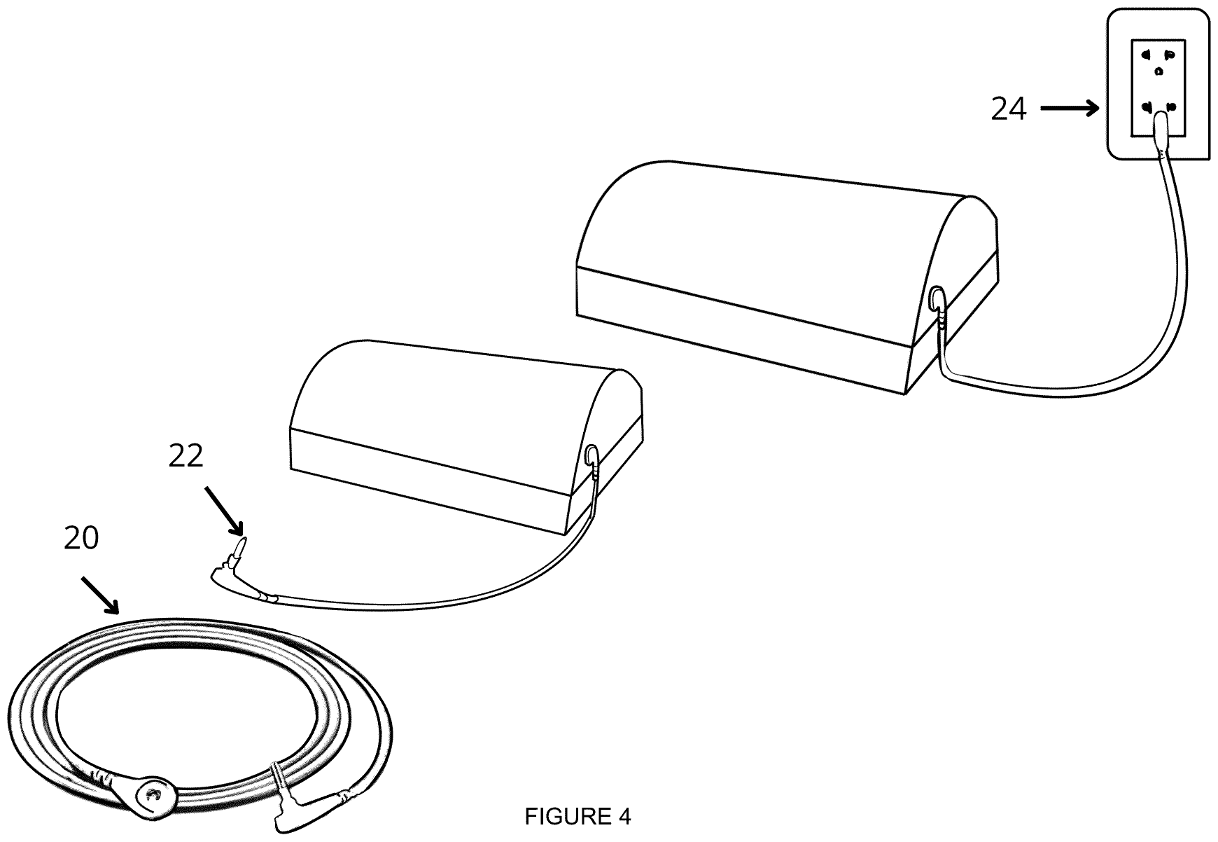

FIG. 5 is a view of the tilt or angle adjustment feature and optional rocking mechanism.

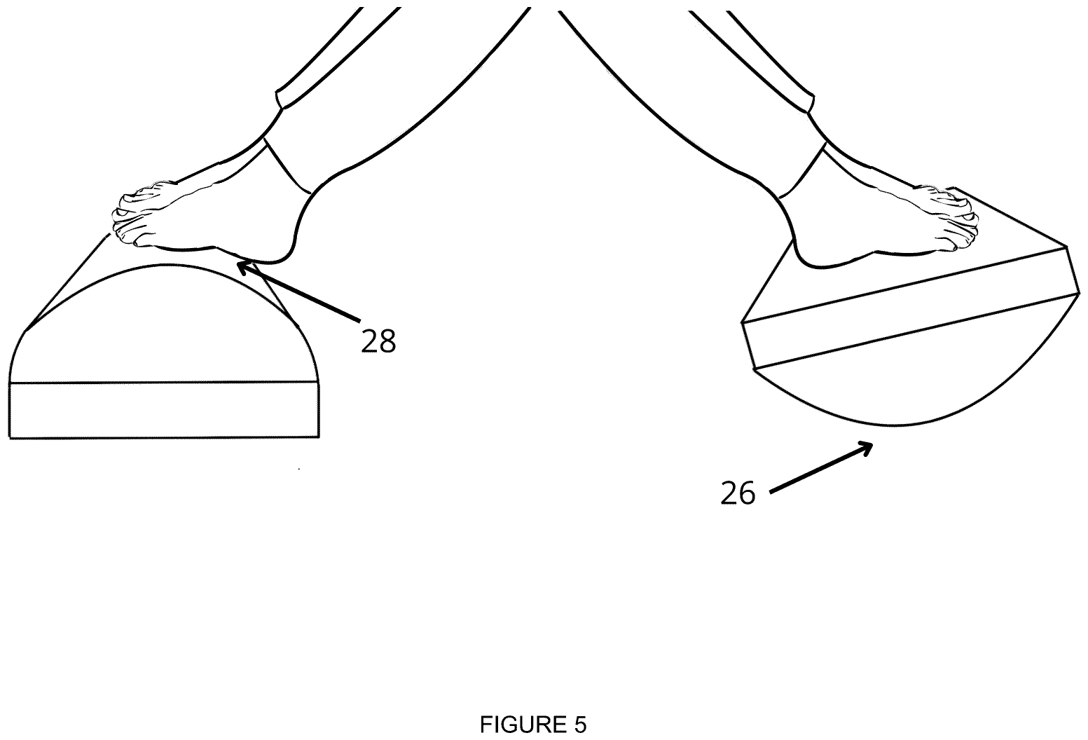

FIG. 6 is a view of the grounding foot rest in use, illustrating user contact with the conductive cover and the grounding connection.

### DETAILED DESCRIPTION OF THE INVENTION

### Ergonomic Design

The grounding foot rest (10) comprises an ergonomic structure configured to support the user's feet in a neutral, comfortable posture. In one embodiment, the structure includes a curved top surface (12) and a rectangular base component (14). The curved surface (12) cradles the user's feet to encourage proper alignment of the ankles, knees, and hips when seated.

An adjustable height mechanism (16) and tilt or angle adjustment feature (28) may be incorporated to accommodate users of different body types and seating arrangements. The design may also enable dynamic movement (26), such as gentle rocking or micro-adjustments, which can help promote circulation and reduce muscle fatigue during extended use.

The foot rest body (10) may be constructed from supportive materials such as high-density foam, memory foam, sponge, other cushioning or resilient structures. A conductive cover (18) wraps the surface that contacts the user's bare feet, allowing for the grounding effect. A handle (30) may be integrated to facilitate portability.

Modular features may allow the curved upper component (12) and the base (14) to be used together or separately, enabling the user to adjust the height, angle, or support configuration to their preference.

2. Grounding Technology A conductive grounding cover (18) is designed to maintain direct contact with the user's skin, establishing a pathway to the Earth's natural electric field. Suitable materials for the cover include silver-threaded fabric, carbon-infused material, or other conductive polymers or fibers.

The grounding cover connects to a detachable grounding wire (20) that attaches the foot rest to a grounding plug (22) compatible with grounded wall outlets (24). Some embodiments may include an adapter or test pen and continuity tester to verify the active grounding connection.

### 3. Versatile Use

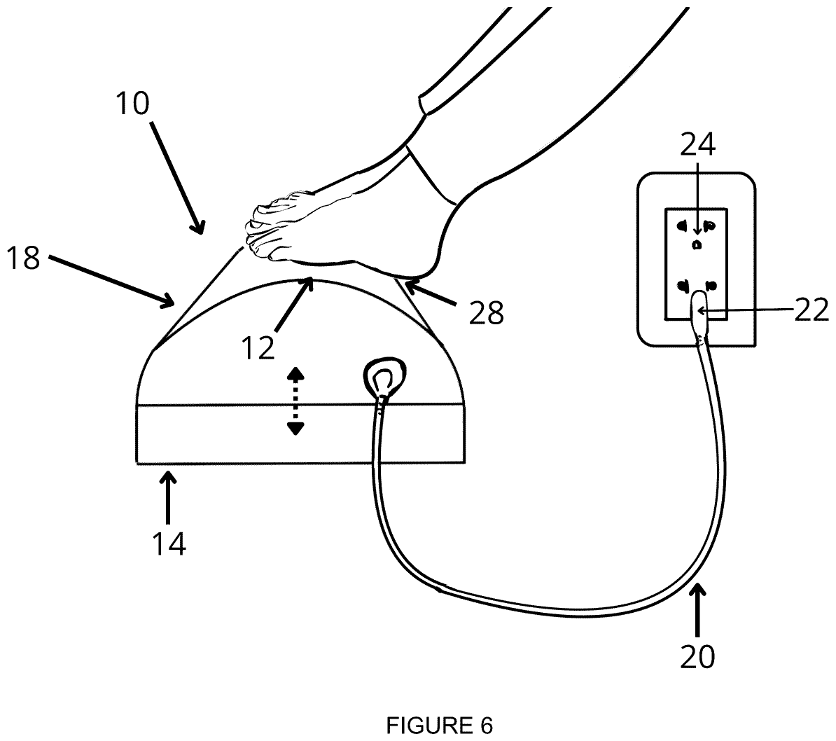

The grounding foot rest (10) is designed for multi-purpose functionality. The main structure may be used under the feet for ergonomic support (28) or separated for use as a lumbar cushion, neck pillow, or leg rest while sitting, working, or traveling. The base component (14) may act as an independent cushion or height adjuster to customize the setup for different environments. As shown in FIG. 6, when in use the user's bare feet rest on the curved top surface (12) in contact with the conductive cover (18), while the grounding wire (20) connects to the grounding source (22).

### 4. Variations and Alternatives

While the above embodiment describes a curved top surface and rectangular base, the invention is not limited to this shape or configuration. The ergonomic structure may be wedge-shaped, rectangular, dome-shaped, flat, or multi-surface, depending on user preference or manufacturing needs. The modular parts may be stackable, foldable, or reconfigurable to adapt to different heights or angles. Materials, dimensions, and contour designs may vary as long as they maintain the essential functions of providing foot support and establishing a grounding pathway. Additional features, such as non-slip bases or removable washable covers, may be included without departing from the scope of the invention.

### 5. Intended Benefits

When used as described, the grounding foot rest combines ergonomic support with a passive connection to the Earth's electric field, offering a practical solution for individuals who spend long hours seated indoors.

1. A grounding foot rest comprising:
a support structure formed of cushioning or supportive material;
a conductive cover configured to facilitate grounding when operatively connected to a grounding source;
a grounding connection mechanism configured to provide a conductive electrical pathway between the conductive cover and a grounding source, including but not limited to a grounded wall outlet, grounding rod, or grounding adapter; and
a configuration adapted to provide foot support and grounding for a user.

2. The grounding foot rest of claim 1, wherein the support structure comprises an ergonomic shape, including a curved, contoured, flat, or otherwise shaped surface to enhance comfort or posture.

3. The grounding foot rest of claim 1, wherein the support structure is adjustable in height, angle, or position by means of one or more built-in mechanisms and/or by comprising two or more modular, detachable, or stackable parts configurable to vary support arrangement.

4. The grounding foot rest of claim 1, wherein the grounding connection mechanism comprises a detachable conductive wire.

5. The grounding foot rest of claim 1, wherein the conductive cover comprises carbon-infused, silver-threaded, or other grounding-capable material.

6. The grounding foot rest of claim 1, wherein the support structure is configurable for portability or alternate use as a pillow, cushion, lumbar support or leg rest.

7. The grounding foot rest of claim 1, wherein the structure enables dynamic foot movement to promote circulation.

8. The grounding foot rest of claim 1, further comprising a test device or continuity tester configured to verify the presence of a grounding connection.
